(19) United States
(12) Patent Application Publication
(10) Pub. No.: US 2007/0282019 A1
(43) Pub. Date: Dec. 6, 2007

(54) PROCESS FOR PRODUCING LIQUEFIED PETROLEUM GAS

(76) Inventors: Kaoru FUJIMOTO, Kitakyushu-shi (JP); Kenji ASAMI, Kitakyushu-shi (JP); Xiaohong LI, Kitakyushu-shi (JP); Qianwen ZHANG, Kitakyushu-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/673,084

(22) Filed: Feb. 9, 2007

(30) Foreign Application Priority Data
Feb. 10, 2006 (JP) 2006-33623

(51) Int. Cl. C07C 27/06

(52) U.S. Cl. 518/715

(57) ABSTRACT

Crude methanol or crude dimethyl ether containing carbon monoxide and/or carbon dioxide is produced from the synthesis gas, which is produced from a carbon-containing starting material such as a natural gas, and then a hydrocarbon containing propane or butane as a main component, i.e., a liquefied petroleum gas is produced from the crude methanol or the crude dimethyl ether.

### TECHNICAL FIELD

This invention relates to a process for producing a liquefied petroleum gas containing propane or butane as a main component from a synthesis gas, via methanol and/or dimethyl ether. This invention also relates to a process for producing a liquefied petroleum gas containing propane or butane as a main component from a carbon-containing starting material such as a natural gas, via a synthesis gas, and methanol and/or dimethyl ether.

### BACKGROUND ART

Liquefied petroleum gas (LPG) is a liquefied petroleum-based or natural-gas-based hydrocarbon which is gaseous at an ambient temperature under an atmospheric pressure by compression while optionally cooling, and the main component of it is propane or butane. LPG is advantageously transportable because it can be stored or transported in a liquid form. Thus, in contrast with a natural gas that requires a pipeline for supply, it has a characteristic that it can be filled in a container to be supplied to any place. For that reason, LPG comprising propane as a main component, i.e., propane gas has been widely used as a fuel for household and business use. At present, propane gas is supplied to about 25 million households (more than 50% of the total households) in Japan. In addition to household and business use, LPG is used as a fuel for a portable product such as a portable gas burner and a disposable lighter (mainly, butane gas), an industrial fuel and an automobile fuel.

Conventionally, LPG has been produced by 1) collection from a wet natural gas, 2) collection from a stabilization (vapor-pressure regulating) process of crude petroleum, 3) separation and extraction of a product in, for example, a petroleum refining process, or the like.

LPG, in particular propane gas used as a household/business fuel, can be expected to be in great demand in the future. Thus, it may be very useful to establish an industrially practicable and new process for producing LPG.

As a process for producing LPG, Japanese Patent Laid-open Publication No. 61-23688 discloses that a synthesis gas consisting of hydrogen and carbon monoxide is reacted in the presence of a mixed catalyst obtained by physically mixing a methanol synthesis catalyst such as a Cu—Zn-based catalyst, a Cr—Zn-based catalyst and a Pd-based catalyst, specifically a CuO—ZnO—Al₂O₃ catalyst or a Pd/SiO₂ catalyst with a methanol conversion catalyst composed of a zeolite having an average pore size of about 10 Å (1 nm) or more, specifically a Y-type zeolite, to give a liquefied petroleum gas or a mixture of hydrocarbons similar in composition to LPG.

Furthermore, as a process for producing LPG, “Selective Synthesis of LPG from Synthesis Gas”, Kaoru Fujimoto et al., Bull. Chem. Soc. Jpn., 58, p. 3059-3060 (1985) discloses that, using a hybrid catalyst consisting of a methanol synthesis catalyst such as a 4 wt % Pd/SiO₂, a Cu—Zn—Al mixed oxide {Cu:Zn:Al=40:23:37 (atomic ratio)} or a Cu-based low-pressure methanol synthesis catalyst (Trade name: BASF S3-85) and a high-silica Y-type zeolite with SiO₂/Al₂O₃=7.6 treated with seam at 450° C. for 1 hour, C2 to C4 paraffins can be produced in a selectivity of 69 to 85% via methanol and dimethyl ether from a synthesis gas.

On the other hand, “Methanol/Dimethyl Ether Conversion on Zeolite Catalysts for Indirect Synthesis of LPG from Natural Gas”, Yingjie Jin et al., Dai 92 Kai Shokubai Touronkai TouronkaiA Yokousyuu, (the summaries of the 92th Catalysis Society of Japan (CatSJ) Meeting, Meeting-A), p. 322, Sep. 18, 2003 discloses a process for producing LPG, using at least one selected from the group consisting of methanol and dimethyl ether as a starting material. Specifically, a starting gas, whose composition is methanol:H₂:N₂=1:1:1, was passed through the two-layered catalyst layer consisting of ZSM-5 as the former layer and Pt—C as the latter layer (ZSM-5/Pt—C Series) or a mixed catalyst layer consisting of ZSM-5 and Pt—C (ZSM-5/Pt—C Pellet-mixture), under a slightly increased pressure, at a reaction temperature of 603 K (330° C.) and at a methanol-based LHSV of 20 h⁻¹, to carry out an LPG production reaction.

Furthermore, “Selective Synthesis of LPG from DME”, Kenji Asami et al., Sekiyugakkai Dai 47 Kai Nenkai Jusyoukouen, Dai 53 Kai Kenkyuhappyoukai Kouenyoushi, (the summaries of the 47th annual meeting of the Japan Petroleum Institute), p. 98-99, May 19, 2004 discloses a process for producing LPG from dimethyl ether and hydrogen by a catalytic reaction. The catalyst used in the process is a hybrid catalyst consisting of a methanol synthesis catalyst and a zeolite (Cu—Zn/USY and the like), Pd ion-exchanged ZSM-5 (Pd-ZSM-5), Pt ion-exchanged ZSM-5 (Pt-ZSM-5), or the like. “Synthesis of LPG from DME with VIIIB Metal Supported on ZSM-5”, Kenji Asami et al., Dai 13 Kai Nihon Energy Gakkai Taikai Kouenyoushisyuu, (the summaries of the 13th meeting of the Japan Institute of Energy), p. 128-129, Jul. 29, 2004 discloses a process for producing LPG from dimethyl ether and hydrogen, using a VIIIB Metal Supported on ZSM-5, specifically Pd ion-exchanged ZSM-5 (Pd-ZSM-5), Pt ion-exchanged ZSM-5 (Pt-ZSM-5), or the like, as a catalyst.

### DISCLOSURE OF THE INVENTION

An objective of this invention is to provide a process for economically producing a hydrocarbon containing propane or butane as a main component, i.e., a liquefied petroleum gas (LPG) from a synthesis gas, via methanol and/or dimethyl ether.

Another objective of this invention is to provide a process for economically producing a hydrocarbon containing propane or butane as a main component, i.e., a liquefied petroleum gas (LPG) from a carbon-containing starting material such as a natural gas, via a synthesis gas, and methanol and/or dimethyl ether.

The present invention provides a process for producing a liquefied petroleum gas, comprising:

(i) a step of producing methanol wherein crude methanol containing methanol, hydrogen and at least one selected from the group consisting of carbon monoxide and carbon dioxide is produced from a synthesis gas using a methanol synthesis catalyst; and

(ii) a step of producing a liquefied petroleum gas wherein a liquefied petroleum gas containing propane or butane as a main component is produced from the crude methanol, which is fed from the step of producing methanol without purification, using a catalyst for producing a liquefied petroleum gas.

Furthermore, the present invention provides a process for producing a liquefied petroleum gas, comprising:

(I) a step of producing a synthesis gas from a carbon-containing starting material and at least one selected from the group consisting of H₂O, O₂ and CO₂;

(II) a step of producing methanol wherein crude methanol containing methanol, hydrogen and at least one selected from the group consisting of carbon monoxide and carbon dioxide is produced from the synthesis gas using a methanol synthesis catalyst; and

(III) a step of producing a liquefied petroleum gas wherein a liquefied petroleum gas containing propane or butane as a main component is produced from the crude methanol, which is fed from the step of producing methanol without purification, using a catalyst for producing a liquefied petroleum gas.

And, the present invention also provides a process for producing a liquefied petroleum gas, comprising:

(i) a step of producing dimethyl ether wherein crude dimethyl ether containing dimethyl ether, hydrogen and at least one selected from the group consisting of carbon monoxide and carbon dioxide is produced from a synthesis gas using a dimethyl ether synthesis catalyst; and

(ii) a step of producing a liquefied petroleum gas wherein a liquefied petroleum gas containing propane or butane as a main component is produced from the crude dimethyl ether, which is fed from the step of producing dimethyl ether without purification, using a catalyst for producing a liquefied petroleum gas.

Furthermore, the present invention provides a process for producing a liquefied petroleum gas, comprising:

(I) a step of producing a synthesis gas from a carbon-containing starting material and at least one selected from the group consisting of H₂O, O₂ and CO₂;

(II) a step of producing dimethyl ether wherein crude dimethyl ether containing dimethyl ether, hydrogen and at least one selected from the group consisting of carbon monoxide and carbon dioxide is produced from the synthesis gas using a dimethyl ether synthesis catalyst; and

(III) a step of producing a liquefied petroleum gas wherein a liquefied petroleum gas containing propane or butane as a main component is produced from the crude dimethyl ether, which is fed from the step of producing dimethyl ether without purification, using a catalyst for producing a liquefied petroleum gas.

Herein, “synthesis gas” means a mixed gas comprising hydrogen and carbon monoxide produced from a carbon-containing starting material including a natural gas and a coal, and is not limited to a mixed gas consisting of hydrogen and carbon monoxide. A synthesis gas may be, for example, a mixed gas comprising carbon dioxide, water, methane, ethane, ethylene and so on. A synthesis gas produced by reforming a natural gas generally contains, in addition to hydrogen and carbon monoxide, carbon dioxide and water vapor.

In this invention, firstly, methanol and/or dimethyl ether is produced from a synthesis gas. Next, methanol and/or dimethyl ether is reacted with hydrogen in the presence of a catalyst for producing a liquefied petroleum gas, to produce a hydrocarbon containing propane or butane as a main component, i.e., a liquefied petroleum gas (LPG). A catalyst for producing a liquefied petroleum gas used in this invention comprises an olefin-hydrogenation catalyst component including Fe, Co, Ni, Cu, Zn, Ru, Rh, Pd, Ir and Pt, and a zeolite. Examples of a catalyst for producing a liquefied petroleum gas include Pd and/or Pt supported on ZSM-5 or USY-type zeolite, and a mixed catalyst comprising a Pd-based catalyst component in which Pd is supported on a support (for example, silica) and a USY-type zeolite.

Methanol is produced from the synthesis gas by reacting carbon monoxide with hydrogen in the presence of a methanol synthesis catalyst. The product of this reaction (crude methanol) generally comprises water; carbon monoxide, which is an unreacted starting material; carbon dioxide and dimethyl ether, which are by-products; and the like.

On the other hand, dimethyl ether is produced from the synthesis gas by reacting carbon monoxide with hydrogen in the presence of a dimethyl ether synthesis catalyst, that is, a mixed catalyst of a methanol synthesis catalyst and a methanol dehydration catalyst. The product of this reaction (crude dimethyl ether) generally comprises water; carbon monoxide, which is an unreacted starting material; carbon dioxide and methanol, which are by-products; and the like.

When constructing a process for producing LPG which comprises the step of producing methanol and/or dimethyl ether from a synthesis gas and the following step of producing LPG from methanol and/or dimethyl ether, it is economically disadvantageous to conduct the purification of a product after methanol and/or dimethyl ether synthesis reaction, because the number of steps increases and a considerable amount of energy is consumed for the purification. It is highly desirable in view of the economics to use crude methanol and/or crude dimethyl ether as a starting gas (a gas fed into a reactor) for producing LPG.

A process for producing an olefin from methanol and/or dimethyl ether has now been well known. The hydrogenation reaction of an olefin into a paraffin is also well known. The combination of these two processes enables the production of LPG from methanol and/or dimethyl ether. More specifically, an olefin containing propylene or butene as a main component is produced from methanol and/or dimethyl ether using a zeolite catalyst; and then the olefin produced is hydrogenated using an olefin-hydrogenation catalyst to form a paraffin containing propane or butane as a main component, i.e., LPG.

In the above process for producing LPG comprising two reaction steps, however, a zeolite catalyst is apt to be deteriorated due to coking in the step of producing an olefin from methanol and/or dimethyl ether. The catalyst, therefore, may not have a sufficiently long catalyst life.

Furthermore, in the above process for producing LPG comprising two reaction steps, a gas containing carbon monoxide and/or carbon dioxide is not preferable as a starting gas. When a starting gas contains carbon monoxide and/or carbon dioxide, in the second step of olefin hydrogenation, carbon monoxide and carbon dioxide may act as a catalyst poisoning component, and thus LPG may not be stably produced for a long period. In addition, the formation of methane by hydrogenation may occur. Accordingly, in the above process for producing LPG comprising two reaction steps, crude methanol and/or crude dimethyl ether, which contains carbon monoxide, carbon dioxide and so on, usually cannot be used as a starting gas for producing LPG.

In contrast, in the process for producing LPG according to this invention, where LPG is produced in one step from methanol and/or dimethyl ether, the presence of carbon monoxide and/or carbon dioxide in a starting gas has no effect on LPG production. Even though using a catalyst comprising an olefin-hydrogenation catalyst component in the LPG production reaction, any problems, which occur in the above process for producing LPG comprising two reaction steps, may not be caused. Consequently, according to this invention, crude methanol and/or crude dimethyl ether, which is produced from a synthesis gas and contains carbon monoxide, carbon dioxide and so on, can be used as a starting gas for producing LPG without treatment.

Furthermore, according to the process for producing LPG of this invention, even when using a zeolite-containing catalyst in the LPG production reaction, deterioration of a zeolite due to coking can be prevented and thus LPG can be stably produced for a long period with reducing a catalyst cost.

As described above, according to this invention, a hydrocarbon containing propane or butane as a main component, i.e., a liquefied petroleum gas (LPG) can be economically produced from a synthesis gas, via methanol and/or dimethyl ether. Moreover, according to this invention, a hydrocarbon containing propane or butane as a main component, i.e., a liquefied petroleum gas (LPG) can be economically produced from a carbon-containing starting material such as a natural gas, via a synthesis gas, and methanol and/or dimethyl ether.

### BRIEF DESCRIPTION OF THE DRAWINGS

FIG. 1 is a process flow diagram showing a main configuration in an example of an LPG producing apparatus suitable for conducting the process for LPG production according to this invention.

### DESCRIPTION OF THE MAIN SYMBOLS

11: a reformer
11a: a reforming catalyst (a catalyst for producing a synthesis gas)
12: a reactor for producing methanol
12a: a methanol synthesis catalyst
13: a reactor for producing a liquefied petroleum gas
13a: a catalyst for producing a liquefied petroleum gas 21, 22, 23, 24: lines.

### BEST MODE FOR CARRYING OUT THE INVENTION

There will be described an embodiment of a process for producing LPG according to this invention with reference to the drawing.

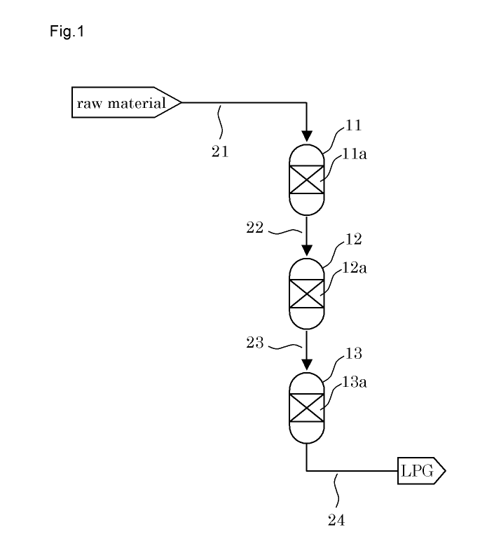

FIG. 1 shows an embodiment of an LPG production apparatus suitable for carrying out a production process for LPG according to this invention.

First, a natural gas (methane) as a carbon-containing starting material is fed into a reformer 11 via a line 21. And, as necessary, oxygen, steam or carbon dioxide is also fed into the line 21 (not shown). In the reformer 11, there is a reforming catalyst layer comprising a reforming catalyst (a catalyst for producing a synthesis gas) 11a. The reformer 11 also has a heating means for supplying heat required for reforming (not shown). In the reformer 11, methane is reformed in the presence of the reforming catalyst to produce a synthesis gas containing hydrogen and carbon monoxide.

The synthesis gas thus produced is fed into a reactor for producing methanol 12 via a line 22. In the reactor 12, there is a catalyst layer comprising a methanol synthesis catalyst 12a. In the reactor 12, crude methanol containing methanol, hydrogen, and carbon monoxide and/or carbon dioxide is produced from the synthesis gas in the presence of the methanol synthesis catalyst.

The crude methanol thus produced is fed into a reactor for producing a liquefied petroleum gas 13 via a line 23 without purification. And, as necessary, hydrogen is also fed into the line 23 (not shown). In the reactor 13, there is a catalyst layer comprising a catalyst for producing a liquefied petroleum gas 13a. In the reactor 13, a hydrocarbon gas containing propane or butane as a main component (a lower-paraffin-containing gas) is produced from the crude methanol in the presence of the catalyst for producing a liquefied petroleum gas.

The hydrocarbon gas thus produced is pressurized and cooled, after optional removal of water, a low-boiling component including hydrogen, and a high-boiling component by, for example, gas-liquid separation, and LPG, which is a product, is obtained from a line 24.

The LPG production apparatus may be, as necessary, provided with a booster, a heat exchanger, a valve, an instrumentation controller and so on, which are not shown.

A dimethyl ether synthesis catalyst can be substituted for a methanol synthesis catalyst 12a. In this case, crude dimethyl ether containing dimethyl ether, hydrogen, and carbon monoxide and/or carbon dioxide is produced from the synthesis gas in the reactor 12, and then a hydrocarbon gas containing propane or butane as a main component (a lower-paraffin-containing gas) is produced from the crude dimethyl ether in the reactor 13.

<Synthesis Gas Production Process>

In a synthesis gas production process, a synthesis gas is generally produced from a carbon-containing starting material and at least one selected from the group consisting of H₂O, O₂ and CO₂.

A carbon-containing substance which can react with at least one selected from the group consisting of H₂O, O₂ and CO₂ to form H₂ and CO, can be used as a carbon-containing starting material. A substance known as a raw material for a synthesis gas can be used as a carbon-containing starting material; for example, lower hydrocarbons such as methane and ethane, a natural gas, a naphtha, a coal, and the like can be used.

Since a catalyst is generally used in a synthesis gas production process, a methanol production process or a dimethyl ether production process, and a liquefied petroleum gas production process in this invention, a carbon-containing starting material (a natural gas, a naphtha, a coal and so on) preferably contains less catalyst poisoning components such as sulfur and a sulfur compound. When a carbon-containing starting material contains a catalyst poisoning component, a step of removing the catalyst poisoning component such as devulcanization can be conducted before a synthesis gas production process, if necessary.

A synthesis gas can be produced by reacting the above carbon-containing starting material with at least one selected from the group consisting of H₂O, O₂ and CO₂ in the presence of a catalyst for producing a synthesis gas (reforming catalyst). A synthesis gas can be produced by a known method, for example, a water-vapor reforming method, a complex reforming method and an autothermal reforming method of a natural gas (methane).

As illustrated in the following formula (1), a preferable composition of a synthesis gas is H₂/CO (molar ratio)=2 in terms of the stoichiometry for methanol production.
CO+2H₂→CH₃OH (1)

When methanol is produced from a synthesis gas mainly, a preferable composition of a synthesis gas produced in a synthesis gas production process is CO:H₂=1:1.5 to 1:2.5 (molar ratio). A ratio of hydrogen to carbon monoxide (H₂/CO; by mole) in a synthesis gas produced is preferably 1.8 or more, more preferably 1.9 or more. A ratio of hydrogen to carbon monoxide (H₂/CO; by mole) in a synthesis gas produced is preferably 2.3 or less, more preferably 2.2 or less. By adjusting a composition of a synthesis gas within the above range, methanol can be produced more efficiently and more economically in the following methanol production process, resulting in more economical production of an LPG.

As illustrated in the following formula (2), a preferable composition of a synthesis gas is H₂/CO (molar ratio)=1 in terms of the stoichiometry for dimethyl ether production.
3CO+3H₂→CH₃OCH₃+CO₂ (2)

When dimethyl ether is produced from a synthesis gas mainly, a preferable composition of a synthesis gas produced in a synthesis gas production process is CO:H₂=1:0.5 to 1:1.5 (molar ratio). A ratio of hydrogen to carbon monoxide (H₂/CO; by mole) in a synthesis gas produced is preferably 0.8 or more, more preferably 0.9 or more. A ratio of hydrogen to carbon monoxide (H₂/CO; by mole) in a synthesis gas produced is preferably 1.2 or less, more preferably 1.1 or less. By adjusting a composition of a synthesis gas within the above range, dimethyl ether can be produced more efficiently and more economically in the following dimethyl ether production process, resulting in more economical production of an LPG.

For example, a shift reactor may be placed downstream of a reformer, which is a reactor for producing a synthesis gas from the above starting materials, so that a synthesis gas composition can be adjusted within the above range by a shift reaction (CO+H₂O→CO₂+H₂).

A synthesis gas having the above composition can be produced by appropriately selecting reaction conditions such as a feeding ratio of a carbon-containing starting material and at least one material selected from the group consisting of water (steam), oxygen and carbon dioxide, a kind of a catalyst for producing a synthesis gas used, and others.

A synthesis gas having the above composition can be produced by the following method, for example.

A synthesis gas used in this invention can be produced by reacting a carbon-containing starting material (specifically, a natural gas and methane), oxygen, carbon dioxide and steam in the presence of a reforming catalyst composed of a composite oxide having a composition represented by the following formula (A), using a gas in which a ratio of (carbon dioxide+steam)/carbon is 0.5 to 3 and a ratio of oxygen/carbon is 0.2 to 1 as a starting gas fed into a reactor, under the operation conditions of a reactor outlet temperature of 900 to 1100° C., and a reaction pressure of 5 to 60 kg/cm².
Ma ·Co_{b}·Ni_{c}·Mg_{d}·Caₑ·O_{f} (A)

wherein M is at least one element selected from Group 6A elements, Group 7A elements, Group 8 transition elements except Co and Ni, Group 1B elements, Group 2B elements, Group 4B elements and lanthanoid elements; and

a, b, c, d and e are the atomic ratios of the respective elements, provided that a+b+c+d+e=1, 0≦a≦0.1, 0.001≦(b+c)≦0.3, 0≦b≦0.3, 0≦c≦0.3, 0.6≦(d+e)≦0.999, 0<d≦0.999, 0≦e≦0.999, and f is a number required for maintaining the charge balance of the respective elements and oxygen.

A ratio of (carbon dioxide+steam)/carbon in a starting gas fed into a reactor is preferably about 0.5 to 2. A reactor outlet temperature is preferably 950 to 1050° C. A reactor outlet pressure is preferably 15 to 20 kg/cm².

A gas space velocity (GHSV) of a starting gas is generally 500 to 200,000 hr⁻¹, preferably 1,000 to 100,000 hr⁻¹, more preferably 1,000 to 70,000 hr⁻¹.

A composite oxide having a composition represented by the above formula (A) is a single-phase solid solution in which MgO and CaO have a rock salt type crystalline structure and a part of Mg atoms and Ca atoms located on the lattice is substituted with Co, Ni or M.

In the above formula (A), M is preferably at least one element selected from the group consisting of manganese, molybdenum, rhodium, ruthenium, platinum, palladium, copper, silver, zinc, tin, lead, lanthanum, and cerium.

The content of M (a) is 0≦a≦0.1, preferably 0≦a≦0.05, more preferably 0≦a≦0.03. The content of M (a) exceeding 0.1 may lead to the lower reforming reaction activity.

The content of Co (b) is 0≦b≦0.3, preferably 0≦b≦0.25, more preferably 0≦b≦0.2. The content of Co (b) exceeding 0.3 may lead to the less prevention effect of carbon precipitation.

The content of Ni (c) is 0≦c≦0.3, preferably 0≦c≦0.25, more preferably 0≦c≦0.2. The content of Ni (c) exceeding 0.3 may lead to the less prevention effect of carbon precipitation.

The total content of Co (b) and Ni (c) (b+c) is 0.001≦(b+c)≦0.3, preferably 0.001≦(b+c)≦0.25, more preferably 0.001≦(b+c)≦0.2. The total content (b+c) exceeding 0.3 may lead to the less prevention effect of carbon precipitation. In addition, the total content (b+c) less than 0.001 may lead to the lower reaction activity.

The total content of Mg (d) and Ca (e) (d+e) is 0.6≦(d+e)≦0.9998, preferably 0.7≦(d+e)≦0.9998, more preferably 0.77≦(d+e)≦0.9998.

Of them, the content of Mg (d) is 0<d≦0.999, preferably 0.2≦d≦0.9998, more preferably 0.37≦d≦0.9998. The content of Ca (e) is 0≦e<0.999, preferably 0≦e≦0.5, more preferably 0≦e≦0.3. This catalyst may not comprise Ca atom.

The total content of Mg (d) and Ca (e) (d+e) depends on the balance among the content of M (a), the content of Co (b) and the content of Ni (c). Any catalyst having (d+e) within the above range, regardless of a ratio of Mg (d) and Ca (e), exhibits the excellent effect in a reforming reaction. A catalyst having higher contents of Ca (e) and M (a) is apt to exhibit the excellent prevention effect of carbon precipitation, and the lower catalytic activity, as compared to a catalyst having a higher content of Mg (d). In the light of activity, it is preferable that the content of Ca (e) is 0.5 or less and the content of M (a) is 0.1 or less.

A preferable reforming catalyst is characterized in that at least one selected from the group consisting of M, Co and Ni are highly dispersed in the composite oxide. Herein, dispersion is defined as the ratio of the number of atoms exposed on the catalyst surface to the number of all atoms of the supported metal. In other words, dispersity is B/A (A:the number of all atoms of Co, Ni and M metal element or compound thereof; B:the number of atoms of Co, Ni and M exposed on the particles). By using a reforming catalyst wherein at least one selected from the group consisting of M, Co and Ni are highly dispersed in the composite oxide, a higher activity can be achieved, and thus may allow the reaction to proceed stoichiometrically, and carbon precipitation can be prevented more efficiently.

The above reforming catalyst can be prepared by an impregnation method, a coprecipitation method, a sol-gel method (hydrolysis method) and a homogeneous precipitation method, for example.

The above reforming catalyst is generally subjected to the activation treatment before use for a synthesis gas production. The activation treatment can be carried out by heating the catalyst at a temperature of 500 to 1000° C., preferably 600 to 1000° C., more preferably 650 to 1000° C. for about 0.5 to 30 hours under a reducing gas such as a hydrogen gas. A reducing gas can be diluted with an inert gas such as a nitrogen gas. The activation treatment can be carried out in a reactor where a reforming reaction is conducted. The catalytic activity is developed by the activation treatment.

In another process for producing a synthesis gas used in this invention, a carbon-containing starting material (specifically, a natural gas and methane) is partially oxidized to form a mixture gas containing an unreacted carbon-containing starting material at a temperature of 600° C. or higher, and then a synthesis gas is produced by reacting the unreacted carbon-containing starting material in the hot mixture gas with carbon dioxide gas and/or steam under increased pressure in the presence of a catalyst in which at least one metal (catalyst metal) selected from rhodium, ruthenium, iridium, palladium and platinum is supported on a support composed of a metal oxide, having a specific surface area of 25 m²/g or less, an electronegativity of a metal ion in the support metal oxide of 13 or less, and the amount of the supported catalyst metal of 0.0005 to 0.1 mol % to the support metal oxide in terms of metal. Alternatively, using a mixture gas consisting of a carbon-containing starting material (specifically, a natural gas and methane), a oxygen-containing gas (air, oxygen and so on), and carbon dioxide gas and/or steam, in the presence of a catalyst in which at least one metal (catalyst metal) selected from rhodium, ruthenium, iridium, palladium and platinum is supported on a support composed of a metal oxide, having a specific surface area of 25 m²/g or less, an electronegativity of a metal ion in the support metal oxide of 13 or less, and the amount of the supported catalyst metal of 0.0005 to 0.1 mol % to the support metal oxide in terms of metal, the carbon-containing starting material in the mixture gas is partially oxidized to form a mixture gas containing an unreacted carbon-containing starting material at a temperature of 600° C. or higher, and simultaneously the unreacted carbon-containing starting material is reacted with carbon dioxide gas and/or steam under increased pressure to produce a synthesis gas.

The catalyst metal may be supported on a support in the form of metal, and may be supported on a support in the form of a metallic compound such as an oxide. The metal oxide used as a support may be a single metal oxide or a composite metal oxide.

The electronegativity of a metal ion in the support metal oxide is 13 or less, preferably 12 or less, more preferably 10 or less. The electronegativity of a metal ion in the support metal oxide exceeding 13 may lead to the considerable carbon precipitation formation when used as a catalyst. Meanwhile, the lower limit of the electronegativity of a metal ion in the support metal oxide is generally about 4.

An electronegativity of a metal ion in the metal oxide is defined by the following equation:
Xi=(1+2i)Xo

wherein Xi represents an electronegativity of the metal ion; Xo represents an electronegativity of the metal; and i represents an electronic number of the metal ion.

When the metal oxide is a composite metal oxide, an average electronegativity of the metal ions is used, and the value is the sum total of electronegativity of the each metal ion in the composite metal oxide multiplied by a mole fraction of each oxide.

An electronegativity of a metal (Xo) is a Pauling's electronegativity. Pauling's electronegativities are listed in Table 15.4 in “Moore Physical Chemistry (latter volume) (4th edition), translated by Ryoichi Fujishiro, Tokyo Kagaku Dojin, p. 707 (1974)”. An electronegativity of a metal ion (Xi) in a metal oxide is detailed in, for example, “Shokubai Koza, Vol. 2, ed. the Catalysis Society of Japan, p. 145 (1985)”.

Examples of the metal oxide include those containing at least one metal such as Mg, Ca, Ba, Zn, Al, Zr and La. Specific examples of such a metal oxide include a single metal oxide such as magnesia (MgO), calcium oxide (CaO), barium oxide (BaO), zinc oxide (ZnO), alumina (Al₂O₃), zirconia (ZrO₂) and lanthanum oxide (La₂O₃); and a composite metal oxide such as MgO/CaO, MgO/BaO, MgO/ZnO, MgO/Al₂O₃, MgO/ZrO₂, CaO/BaO, CaO/ZnO, CaO/Al₂O₃, CaO/ZrO₂, BaO/ZnO, BaO/Al₂O₃, BaO/ZrO₂, ZnO/Al₂O₃, ZnO/ZrO₂, Al₂O₃/ZrO₂, La₂O₃/MgO, La₂O₃/Al₂O₃ and La₂O₃/CaO.

The specific surface area of the used catalyst is 25 m²/g or less, preferably 20 m²/g or less, more preferably 15 m²/g or less, particularly preferably 10 m²/g or less. Meanwhile, the lower limit of the specific surface area of the used catalyst is generally about 0.01 m²/g. By adjusting the specific surface area of the catalyst within the above range, a carbon precipitation forming activity of the catalyst can be more sufficiently reduced.

In the catalyst used in this process, the specific surface area of the catalyst is substantially equivalent to the specific surface area of the support metal oxide. Accordingly, the specific surface area of the support metal oxide is 25 m²/g or less, preferably 20 m²/g or less, more preferably 15 m²/g or less, particularly preferably 10 m²/g or less. Meanwhile, the lower limit of the specific surface area of the support metal oxide is generally about 0.01 m²/g.

In this invention, a specific surface area of a catalyst and a support metal oxide can be determined at a temperature of 15° C. by a BET method.

A catalyst having a specific surface area of 25 m²/g or less can be prepared by calcining a metal oxide, which is a support, at a temperature of 300 to 1300° C., preferably 650 to 1200° C., prior to supporting a catalyst metal on the support, and further calcining a catalyst metal supported on metal oxide at a temperature of 600 to 1300° C., preferably 650 to 1200° C., after supporting a catalyst metal on the support. Alternatively, a catalyst having a specific surface area of 25 m²/g or less can be prepared by calcining a catalyst metal supported on metal oxide at a temperature of 600 to 1300° C., preferably 650 to 1200° C., after supporting a catalyst metal on a metal oxide, which is a support. A specific surface area of the obtained catalyst and a specific surface area of the support metal oxide can be controlled by controlling a calcination temperature and a calcination time.

The amount of the supported catalyst metal is 0.0005 to 0.1 mol % to the support metal oxide in terms of metal. The amount of the supported catalyst metal is preferably 0.001 mol % or more, more preferably 0.002 mol % or more, to the support metal oxide in terms of metal. In addition, the amount of the supported catalyst metal is preferably 0.09 mol % or less to the support metal oxide in terms of metal.

The above catalyst has a sufficiently high activity of forming a synthesis gas from a carbon-containing starting material and a remarkably low activity of forming a carbon precipitation, due to the small specific surface area of the catalyst and the extremely small amount of the supported catalyst metal.

The above catalyst can be prepared in accordance with a known method. For example, a salt of catalyst metal or an aqueous solution thereof is added to a suspension in which a support metal oxide is dispersed in water, and mixed, and then a catalyst metal supported on metal oxide is separated from the resulting solution, dried and calcined to give a catalyst (impregnation method). Alternatively, a pore volume of solution containing a salt of catalyst metal is added drop by drop to an evacuated support metal oxide, while keeping the whole surface of the support wet, and then the resulting catalyst metal supported on metal oxide is dried and calcined to give a catalyst (incipient-wetness method).

A carbon-containing starting material (specifically, a natural gas and methane) can be reacted with steam (water vapor) and/or carbon dioxide in the presence of the above catalyst to produce a synthesis gas used in this invention.

In a process in which a carbon-containing starting material is reacted with carbon dioxide (CO₂ reforming), a reaction temperature is 500 to 1200° C., preferably 600 to 1000° C., and a reaction pressure is 5 to 40 kg/cm²G, preferably 5 to 30 kg/cm²G. When the CO₂ reforming reaction is conducted with a fixed bed, a gas space velocity (GHSV) is 1,000 to 10,000 hr⁻¹, preferably 2,000 to 8,000 hr⁻¹. A content of CO₂ in a starting gas fed into a reactor is 20 to 0.5 moles, preferably 10 to 1 moles of CO₂ per one mole of carbon in the carbon-containing starting material.

In a process in which a carbon-containing starting material is reacted with steam (steam reforming), a reaction temperature is 600 to 1200° C., preferably 600 to 1000° C., and a reaction pressure is 1 to 40 kg/cm²G, preferably 5 to 30 kg/cm²G. When the steam reforming reaction is conducted with a fixed bed, a gas space velocity (GHSV) is 1,000 to 10,000 hr⁻¹, preferably 2,000 to 8,000 hr⁻¹. A content of steam in a starting gas fed into a reactor is 0.5 to 20 moles, preferably 1 to 10 moles, further preferably 1 to 1.5 moles of steam (H₂O) per one mole of carbon in the carbon-containing starting material.

When a carbon-containing starting material is reacted with a mixture of steam and CO₂ to produce a synthesis gas, there are no restrictions to a ratio of steam to CO₂, but a ratio of H₂O/CO₂ (molar ratio) is generally 0.1 to 10.

In this process for producing a synthesis gas, the energy required for the above reforming reaction is supplied by combustion heat generated by partially oxidation (partially combustion) of a carbon-containing starting material, which is a starting material for reforming.

The partially oxidation reaction of a carbon-containing starting material is generally carried out at a reaction temperature of 600 to 1500° C., preferably 700 to 1300° C., and under a reaction pressure of 5 to 50 kg/cm²G, preferably 10 to 40 kg/cm²G. Oxygen is used as an oxidizing agent for partially oxidizing a carbon-containing starting material. As the source of oxygen, an oxygen-containing gas including air and oxygen-enriched air, as well as pure oxygen, can be used. In regard to a content of oxygen in a starting gas fed into a reactor, an atomic ratio of oxygen to carbon in a carbon-containing starting material (O/C) is 0.1 to 4, preferably 0.5 to 2.

A mixture gas containing an unreacted carbon-containing starting material at a temperature of 600° C. or higher, preferably 700 to 1300° C., can be obtained by the above partially oxidation of a carbon-containing starting material. And a synthesis gas can be produced by reacting the unreacted carbon-containing starting material in the resulting mixture gas with carbon dioxide and/or steam under the above-mentioned conditions. Carbon dioxide and/or steam may be added to the mixture gas obtained by the partially oxidation of a carbon-containing starting material. Alternatively, carbon dioxide and/or steam may be added to and mixed with the carbon-containing starting material to be subjected to the partially oxidation reaction previously. In the latter case, the partially oxidation reaction of a carbon-containing starting material and the reforming reaction may proceed simultaneously.

A reforming reaction of a carbon-containing starting material can be conducted in various types of reactors, but is preferably conducted in a fixed bed or a fluidized bed.

<Methanol-Dimethyl Ether Production Process>

In this invention, carbon monoxide is reacted with hydrogen in the presence of a catalyst, to produce crude methanol containing methanol, hydrogen, and carbon monoxide and/or carbon dioxide, or crude dimethyl ether containing dimethyl ether, hydrogen, and carbon monoxide and/or carbon dioxide, from the synthesis gas obtained in the above synthesis gas production process.

Next, there will be described this process for producing methanol mainly from the synthesis gas (Methanol production process) and process for producing dimethyl ether mainly from the synthesis gas (Dimethyl ether production process)

<Methanol Production Process>

In the methanol production process, methanol (crude methanol) is produced from the synthesis gas obtained in the above synthesis gas production process by reacting carbon monoxide with hydrogen in the presence of a methanol synthesis catalyst. The crude methanol produced in this process comprises carbon monoxide, which is an unreacted starting material, and carbon dioxide, which is a by-product.

A gas fed into a reactor in a methanol production process may be a gas obtained by separating a certain component such as water and carbon dioxide from the synthesis gas produced in the above synthesis gas production process. The water and carbon dioxide separated from the synthesis gas can be recycled to the synthesis gas production step.

In this methanol production process, methanol can be produced in accordance with a known method. For example, methanol can be produced by the following method.

Methanol synthesis can be carried out by a vapor phase reaction, a liquid phase reaction in which a methanol synthesis catalyst is dispersed in an inert solvent, or the like. In the liquid phase reaction (slurry process), a petroleum solvent may be used as a solvent, and the amount of the methanol synthesis catalyst to be used may be, for example, about 25 to 50% by weight.

A fixed-bed contact synthesis reactor may be selected from, for example, a quench type reactor, a multitubular type reactor, a multistage type reactor, a multistage cooling radial flow type reactor, a double pipe heat exchange type reactor, an internal cooling coil type reactor, and a mixed flow type reactor.

Examples of a methanol synthesis catalyst include any of methanol synthesis catalysts known in the art; specifically, Cu—Zn-based catalysts such as copper oxide-zinc oxide, copper oxide-zinc oxide-aluminum oxide (alumina) and copper oxide-zinc oxide-chromium oxide; Zn—Cr-based catalysts such as zinc oxide-chromium oxide and zinc oxide-chromium oxide-alumina; and Cu—ZnO-based catalysts. An example of a methanol synthesis catalyst having a high durability under an atmosphere of relatively high concentration of carbon dioxide is an oxide containing Cu, Zn, Al, Ga and M (at least one element selected from alkaline earth metals and rare earth elements) at a ratio of Cu:Zn:Al:Ga:M=100:10-200:1-20:1-20:0.1-20 (atomic ratio).

The methanol synthesis catalyst may comprise an additive component as long as the intended effect would not be impaired.

A gas fed into a reactor is preferably a synthesis gas containing carbon monoxide and hydrogen at a ratio of CO:H₂=1:1.5 to 1:2.5 (molar ratio). A ratio of hydrogen to carbon monoxide (H₂/CO; by mole) in a gas fed into a reactor is more preferably 1.8 or more, particularly preferably 1.9 or more. A ratio of hydrogen to carbon monoxide (H₂/CO; by mole) in a gas fed into a reactor is more preferably 2.3 or less, particularly preferably 2.2 or less.

A gas fed into a reactor may contain a component other than carbon monoxide and hydrogen. In some cases, it is preferable that a gas fed into a reactor contains carbon dioxide. A content of carbon dioxide in a gas fed into a reactor may be 0.1 to 15 mol %, for example.

When using a Cu—Zn-based catalyst as a methanol synthesis catalyst, a reaction temperature may be about 200 to 300° C. and a reaction pressure may be about 1 to 10 MPa.

When using a Zn—Cr-based catalyst as a methanol synthesis catalyst, a reaction temperature may be about 250 to 400° C. and a reaction pressure may be about 10 to 60 MPa.

The reaction conditions such as a reaction temperature and a reaction pressure are not limited to the above ranges, and can be appropriately determined, depending on a kind of a catalyst to be used, and the like.

The crude methanol thus produced generally comprises, in addition to methanol, carbon monoxide and hydrogen, which are unreacted starting materials, carbon dioxide, water, dimethyl ether and so on. In this invention, the crude methanol is used, without purification, as a starting gas for the following step of producing a liquefied petroleum gas.

<Dimethyl Ether Production Process>

In the dimethyl ether production process, dimethyl ether (crude dimethyl ether) is produced from the synthesis gas obtained in the above synthesis gas production process by reacting carbon monoxide with hydrogen in the presence of a dimethyl ether synthesis catalyst. The crude dimethyl ether produced in this process comprises carbon monoxide, which is an unreacted starting material, and carbon dioxide, which is a by-product.

A gas fed into a reactor in a dimethyl ether production process may be a gas obtained by separating a certain component from the synthesis gas produced in the above synthesis gas production process. Generally, water is separated from the synthesis gas by, for example, gas-liquid separation with cooling, and then carbon dioxide is separated from the resulting gas by, for example, gas-liquid separation with cooling or absorption separation by an amine or the like, and then the resulting gas is fed into the reactor. The water and carbon dioxide separated from the synthesis gas can be recycled to the synthesis gas production step.

In this dimethyl ether production process, dimethyl ether can be produced in accordance with a known method. For example, dimethyl ether can be produced by the following method.

A dimethyl ether synthesis reaction can be conducted in various types of reactors such as a fixed-bed type reactor, a fluid-bed type reactor and a slurry-bed type reactor, and is generally preferably conducted in a slurry-bed type reactor. When using a slurry type reactor, the temperature in the reactor is more uniform and the production amount of the by-product(s) is reduced.

Examples of a dimethyl ether synthesis catalyst include a catalyst comprising at least one methanol synthesis catalyst component and at least one methanol dehydration catalyst component, and a catalyst comprising at least one methanol synthesis catalyst component, at least one methanol dehydration catalyst component and at least one water gas shift catalyst component.

Herein, a “methanol synthesis catalyst component” means a compound which can act as a catalyst in the reaction of CO+2H₂→CH₃OH. And a “methanol dehydration catalyst component” means a compound which can act as a catalyst in the reaction of 2CH₃OH→CH₃OCH₃+H₂O. And a “water gas shift catalyst component” means a compound which can act as a catalyst in the reaction of CO+H₂O→H₂+CO₂.

Examples of a methanol synthesis catalyst component include any of known methanol synthesis catalysts; specifically, copper oxide-zinc oxide, zinc oxide-chromium oxide, copper oxide-zinc oxide-chromium oxide, copper oxide-zinc oxide-alumina, and zinc oxide-chromium oxide-alumina. In a copper oxide-zinc oxide catalyst and a copper oxide-zinc oxide-alumina catalyst, a ratio of zinc oxide to copper oxide (zinc oxide/copper oxide; by weight) is about 0.05 to 20, more preferably about 0.1 to 5, and a ratio of alumina to copper oxide (alumina/copper oxide; by weight) is about 0 to 2, more preferably about 0 to 1. In a zinc oxide-chromium oxide catalyst and a zinc oxide-chromium oxide-alumina catalyst, a ratio of chromium oxide to zinc oxide (chromium oxide/zinc oxide; by weight) is about 0.1 to 10, more preferably about 0.5 to 5, and a ratio of alumina to zinc oxide (alumina/zinc oxide; by weight) is about 0 to 2, more preferably about 0 to 1.

Generally, a methanol synthesis catalyst component can act as a catalyst in the reaction of CO+H₂O→H₂+CO₂, and it also serves as a water gas shift catalyst component.

Examples of a methanol dehydration catalyst component include γ-alumina, silica, silica-alumina and zeolite, which are acid-base catalysts. Examples of a metal oxide component in zeolite include alkali metal oxides such as sodium oxide and potassium oxide, and alkaline earth metal oxides such as calcium oxide and magnesium oxide.

Examples of a water gas shift catalyst component include copper oxide-zinc oxide, and iron oxide-chromium oxide. In a copper oxide-zinc oxide catalyst, a ratio of copper oxide to zinc oxide (copper oxide/zinc oxide; by weight) is about 0.1 to 20, more preferably about 0.5 to 10. In a iron oxide-chromium oxide catalyst, a ratio of chromium oxide to iron oxide (chromium oxide/iron oxide; by weight) is about 0.1 to 20, more preferably about 0.5 to 10. Examples of a water gas shift catalyst component which also serves as a methanol dehydration catalyst component include copper (including copper oxide)-alumina.

There are no restrictions to a ratio of a methanol synthesis catalyst component/a methanol dehydration catalyst component/a water gas shift catalyst component, which can be appropriately determined, depending on kinds of catalyst components, reaction conditions, and the like. Generally, a ratio of a methanol dehydration catalyst component to a methanol synthesis catalyst component (a methanol dehydration catalyst component/a methanol synthesis catalyst component; by weight) is about 0.1 to 5, more preferably about 0.2 to 2. A ratio of a water gas shift catalyst component to a methanol synthesis catalyst component (a water gas shift catalyst component/a methanol synthesis catalyst component; by weight) is about 0.2 to 5, more preferably about 0.5 to 3. When a methanol synthesis catalyst component also acts as a water gas shift catalyst component, it is preferable that the content of the methanol synthesis catalyst component is the sum of the above contents of the methanol synthesis catalyst component and of the water gas shift catalyst component.

A dimethyl ether synthesis catalyst is preferably a mixture of a methanol synthesis catalyst component, a methanol dehydration catalyst component and, if necessary, a water gas shift catalyst component. The mixture may be, if necessary, molded after mixing these catalyst components homogeneously. After molding, the resulting catalyst may be re-pulverized. The better catalyst performance may be achieved when using a catalyst obtained by mixing the catalyst components homogeneously, pressing the mixture, and then pulverizing the resulting catalyst.

When using a slurry type reactor, average particle sizes of a methanol synthesis catalyst component, a methanol dehydration catalyst component and a water gas shift catalyst component are preferably 300 μm or less, more preferably 1 to 200 μm, particularly preferably 10 to 150 μm.

The dimethyl ether synthesis catalyst may comprise an additive component as long as the intended effect would not be impaired.

In the dimethyl ether production process, dimethyl ether is produced by reacting carbon monoxide with hydrogen using the above dimethyl ether synthesis catalyst.

As described above, the reaction is preferably conducted in a slurry-bed type reactor.

When using a slurry type reactor, a dimethyl ether synthesis catalyst is used as a slurry catalyst wherein the dimethyl ether synthesis catalyst is dispersed in an oily medium as a solvent.

The oily medium is required to be kept in a liquid state stably under the reaction conditions. Examples of an oily medium include aliphatic, aromatic and alicyclic hydrocarbons, alcohols, ethers, esters, ketones and halides thereof. The oily medium may be used alone or in combination of two or more. A preferable oily medium is a medium consisting essentially of one or more hydrocarbons. A desulfurized light oil, a vacuum light oil, a high-boiling fraction of hydrogenated coal tar, a Fischer-Tropsch synthetic oil, and a high-boiling edible oil can be used as an oily medium.

The amount of the dimethyl ether synthesis catalyst to be used can be appropriately determined, depending on a kind of a solvent (oily medium) to be used, reaction conditions, and the like. Generally, the amount is preferably about 1 to 50 wt % to the solvent. The amount of the dimethyl ether synthesis catalyst to be used is more preferably 5 wt % or more, particularly preferably 10 wt % or more to the solvent. On the other hand, the amount of the dimethyl ether synthesis catalyst to be used is more preferably 40 wt % or less to the solvent.

A gas fed into a reactor is preferably a synthesis gas containing carbon monoxide and hydrogen at a ratio of CO:H₂=1:0.5 to 1:1.5 (molar ratio). A ratio of hydrogen to carbon monoxide (H₂/CO; by mole) in a gas fed into a reactor is more preferably 0.8 or more, particularly preferably 0.9 or more. A ratio of hydrogen to carbon monoxide (H₂/CO; by mole) in a gas fed into a reactor is more preferably 1.2 or less, particularly preferably 1.1 or less.

A gas fed into a reactor may contain a component other than carbon monoxide and hydrogen.

When using a slurry type reactor, a reaction temperature is preferably 150 to 400° C., more preferably 200° C. or higher and 350° C. or lower. By controlling a reaction temperature within the above range, a higher conversion of carbon monoxide can be achieved.

A reaction pressure is preferably 1 to 30 MPa, more preferably 2 MPa or higher and 8 MPa or lower. By controlling a reaction pressure to be 1 MPa or higher, a higher conversion of carbon monoxide can be achieved. Meanwhile, in the light of economical efficiency, a reaction pressure is preferably 30 MPa or lower.

A space velocity (a feed rate of a starting gas in normal state per 1 kg of a catalyst) is preferably 100 to 50000 L/kg·h, more preferably 500 L/kg·h or higher and 30000 L/kg·h or lower. By controlling a space velocity to be 50000 L/kg·h or lower, a higher conversion of carbon monoxide can be achieved. Meanwhile, in the light of economical efficiency, a space velocity is preferably 100 L/kg·h or higher.

The crude dimethyl ether thus produced generally comprises, in addition to dimethyl ether, carbon monoxide and hydrogen, which are unreacted starting materials, carbon dioxide, water, methanol and so on. In this invention, the crude dimethyl ether is used, without purification, as a starting gas for the following step of producing a liquefied petroleum gas.

<Liquefied Petroleum Gas Production Process>

In the liquefied petroleum gas production process, a lower-paraffin-containing gas containing propane or butane as a main component is produced from the crude methanol obtained in the above methanol production process, mainly by reacting methanol with hydrogen in the presence of a catalyst for producing a liquefied petroleum gas. Alternatively, a lower-paraffin-containing gas containing propane or butane as a main component is produced from the crude dimethyl ether obtained in the above dimethyl ether production process, mainly by reacting dimethyl ether with hydrogen in the presence of a catalyst for producing a liquefied petroleum gas. When the crude methanol obtained in the methanol production process or the crude dimethyl ether obtained in the dimethyl ether production process does not contain a sufficient amount of hydrogen for the LPG synthesis reaction, hydrogen may be added to the crude methanol or the crude dimethyl ether. And then, water, a low-boiling component such as hydrogen, and a high-boiling component is separated from the lower-paraffin-containing gas produced, as necessary, to obtain a liquefied petroleum gas (LPG). If necessary, the gas may be pressurized and/or cooled so as to obtain a liquefied petroleum gas.

Examples of a catalyst for producing a liquefied petroleum gas include a catalyst comprising an olefin-hydrogenation catalyst component and a zeolite; for example, a catalyst in which an olefin-hydrogenation catalyst component is supported on a zeolite; and a mixed catalyst comprising a zeolite and a catalyst component in which an olefin-hydrogenation catalyst component is supported on a support such as silica. Other examples of a catalyst for producing a liquefied petroleum gas include a catalyst comprising a methanol synthesis catalyst and a zeolite; specifically, a catalyst comprising a Cu—Zn-based methanol synthesis catalyst and a USY-type zeolite in a ratio of the Cu—Zn-based methanol synthesis catalyst:the USY-type zeolite=1:5 to 2:1 (by weight); a catalyst comprising a Cu—Zn-based methanol synthesis catalyst and β-zeolite in a ratio of the Cu—Zn-based methanol synthesis catalyst:the β-zeolite=1:5 to 2:1 (by weight); and a catalyst comprising a Pd-based methanol synthesis catalyst and a β-zeolite in a ratio of the Pd-based methanol synthesis catalyst:the β-zeolite=1:5 to 2.5:1 (by weight).

Herein, an “olefin-hydrogenation catalyst component” means a compound which can act as a catalyst in a hydrogenation reaction of an olefin into a paraffin. Specific examples of an olefin-hydrogenation catalyst component include Fe, Co, Ni, Cu, Zn, Ru, Rh, Pd, Ir, Pt and so on. And a “methanol synthesis catalyst” means a compound which can act as a catalyst in the reaction of CO+2H₂→CH₃OH. In the above catalyst comprising a methanol synthesis catalyst and a zeolite, the methanol synthesis catalyst acts as an olefin-hydrogenation catalyst component. And a zeolite is those which can act as a catalyst in a condensation reaction of methanol into a hydrocarbon and/or a condensation reaction of dimethyl ether into a hydrocarbon.

In the liquefied petroleum gas production process, paraffin containing propane or butane as a main component (LPG) may be produced from at least one selected from the group consisting of methanol and dimethyl ether, and hydrogen, following the formula (3) shown below.

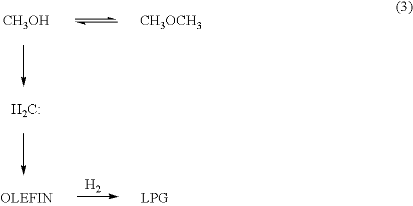

In this invention, methanol is dehydrated to generate a carbene (H₂C:) by a concerted catalysis of an acidic site and a basic site, which are at a spatial field inside a pore in a zeolite. And then, the carbene is polymerized to form an olefin containing propylene or butene as a main component. More specifically, it may be thought that ethylene is formed as a dimer; propylene is formed as a trimer or a reaction product with ethylene; and butylene is formed as a tetramer, a reaction product with propylene or a product of dimerization of ethylene.

In the olefin formation process, there would occur other reactions such as formation of dimethyl ether by dehydration-dimerization of methanol and formation of methanol by hydration of dimethyl ether.

And then, the formed olefin is hydrogenated by the catalysis of an olefin-hydrogenation catalyst component, to form a paraffin containing propane or butane as a main component, i.e., LPG.

For the above catalyst, any of Cu—Zn-based methanol synthesis catalysts known in the art can be used. Meanwhile, examples of a Pd-based methanol synthesis catalyst include a catalyst in which 0.1 to 10 wt % Pd is supported on a support such as silica; and a catalyst in which 0.1 to 10 wt % Pd and 5 wt % or less (excluding 0 wt %) at least one selected from the group consisting of alkali metals, alkaline earth metals and lanthanoid metals such as Ca are supported on a support such as silica.

Among them, a particularly preferable catalyst for producing a liquefied petroleum gas is a catalyst in which an olefin-hydrogenation catalyst component is supported on a zeolite; and a mixed catalyst comprising a zeolite and a catalyst component in which an olefin-hydrogenation catalyst component is supported on a support such as silica.

Specific examples of an olefin-hydrogenation catalyst component include Fe, Co, Ni, Cu, Zn, Ru, Rh, Pd, Ir, Pt and so on. The olefin-hydrogenation catalyst components may be used alone or in combination of two or more.

Among them, a preferable olefin-hydrogenation catalyst component is Pd or Pt, more preferably Pd. By using Pd and/or Pt as an olefin-hydrogenation catalyst component, the production amount of carbon monoxide and carbon dioxide as by-products can be more sufficiently reduced, while maintaining a high yield of propane and butane.

Pd and Pt may not be necessarily contained as a metal, but can be contained in the form of an oxide, a nitrate, a chloride or the like. In such a case, it is preferred that the catalyst may be subjected to, for example, reduction by hydrogen before the reaction, to convert Pd and/or Pt into metallic palladium and/or metallic platinum, for achieving higher catalytic activity.

The reduction treatment condition for activating Pd and/or Pt can be determined, depending on some factors such as the types of a supported palladium compound and/or a supported platinum compound, as appropriate.

A preferable catalyst in which an olefin-hydrogenation catalyst component is supported on a zeolite is Pd and/or Pt supported ZSM-5 or USY-type zeolite, more preferably Pd supported ZSM-5 or USY-type zeolite. By using ZSM-5 or USY-type zeolite as a zeolite for a support of Pd and/or Pt, a higher catalytic activity and a higher yield of propane and butane can be achieved, and furthermore the production amount of carbon monoxide and carbon dioxide as by-products can be more sufficiently reduced.

In the light of catalytic activity, Pd and/or Pt is preferably supported on a ZSM-5 or USY-type zeolite in a highly dispersed manner.

In this catalyst for producing a liquefied petroleum gas, the total amount of supported Pd and/or Pt is preferably 0.005 wt % or more, more preferably 0.01 wt % or more, particularly preferably 0.05 wt % or more, in the light of achieving a higher selectivity. And, the total amount of supported Pd and/or Pt is preferably 5 wt % or less, more preferably 1 wt % or less, particularly preferably 0.7 wt % or less, in the light of catalytic activity, dispersity and economical efficiency. By adjusting the total amount of supported Pd and/or Pt in a catalyst for producing a liquefied petroleum gas within the above range, propane and/or butane can be produced with a higher conversion, a higher selectivity and a higher yield.

A preferable ZSM-5, on which an olefin-hydrogenation catalyst component is supported, is high-silica ZSM-5, more specifically ZSM-5 with a Si/Al ratio (atomic ratio) of 20 to 100. By using ZSM-5 with a Si/Al ratio (atomic ratio) of 20 to 100, a higher catalytic activity and a higher yield of propane and butane can be achieved, and furthermore the production amount of carbon monoxide and carbon dioxide as by-products can be more sufficiently reduced. A Si/Al ratio (atomic ratio) of ZSM-5 is more preferably 70 or less, particularly preferably 60 or less.

A SiO₂/Al₂O₃ ratio of a USY-type zeolite is more preferably 5 or more, particularly preferably 15 or more. And, a SiO₂/Al₂O₃ ratio of a USY-type zeolite is preferably 70 or less, more preferably 50 or less, particularly preferably 40 or less, further preferably 25 or less.

The above catalyst for producing a liquefied petroleum gas may be a catalyst in which other components, in addition to Pd and/or Pt, are supported on ZSM-5 or USY-type zeolite, as long as the desired effects of the catalyst would not be impaired.

A catalyst for producing a liquefied petroleum gas in which an olefin-hydrogenation catalyst component is supported on a zeolite can be prepared by a known method such as an ion exchange method and an impregnation method. Sometimes, in comparison with a catalyst for producing a liquefied petroleum gas prepared by an impregnation method, a catalyst for producing a liquefied petroleum gas prepared by an ion exchange method may exhibit a higher catalytic activity, and thus may allow an LPG production reaction to proceed at a lower reaction temperature, and a higher selectivity for a hydrocarbon and a higher selectivity for propane and butane may be achieved.

A catalyst in which an olefin-hydrogenation catalyst component is supported on zeolite may be used, if necessary, after pulverization or molding. A molding method of a catalyst is not particularly limited, but is preferably a dry method including an extrusion and a tablet-compression.

A preferable mixed catalyst comprising a zeolite and a catalyst component in which an olefin-hydrogenation catalyst component is supported on a support is a mixed catalyst comprising a Pd-based catalyst component in which Pd is supported on a support and a USY-type zeolite. By using a USY-type zeolite, a higher catalytic activity and a higher yield of propane and butane can be achieved, and furthermore the production amount of carbon monoxide and carbon dioxide as by-products can be more sufficiently reduced.

A ratio of the Pd-based catalyst component to the USY-type zeolite (Pd-based catalyst component/USY-type zeolite; by weight) is preferably 0.1 or more, more preferably 0.3 or more. By adjusting a ratio of the Pd-based catalyst component to the USY-type zeolite (Pd-based catalyst component/USY-type zeolite; by weight) to 0.1 or more, a higher yield of LPG can be achieved.

A ratio of the Pd-based catalyst component to the USY-type zeolite (Pd-based catalyst component/USY-type zeolite; by weight) is preferably 1.5 or less, more preferably 1.2 or less, particularly preferably 0.8 or less. By adjusting a ratio of the Pd-based catalyst component to the USY-type zeolite (Pd-based catalyst component/USY-type zeolite; by weight) to 1.5 or less, a higher yield of LPG can be achieved, and furthermore the production amount of carbon monoxide, carbon dioxide and methane as by-products can be more sufficiently reduced. Moreover, by adjusting a ratio of the Pd-based catalyst component to the USY-type zeolite (Pd-based catalyst component/USY-type zeolite; by weight) to 0.8 or less, a further higher yield of LPG can be achieved, and the production amount of heavy hydrocarbons (C5 or more) as by-products can be more sufficiently reduced.

A ratio of the Pd-based catalyst component to the USY-type zeolite is not limited to the above range, and can be appropriately determined, depending on the amount of Pd in the Pd-based catalyst component, and the like.

A Pd-based catalyst component is Pd supported on a support. In the light of catalytic activity, Pd is preferably supported on a support in a highly dispersed manner.

The amount of supported Pd in a Pd-based catalyst component is preferably 0.1 wt % or more, more preferably 0.3 wt % or more. In the light of dispersibility and economical efficiency, the amount of supported Pd in a Pd-based catalyst component is preferably 5 wt % or less, more preferably 3 wt % or less. By adjusting the amount of supported Pd in a Pd-based catalyst component within the above range, propane and/or butane can be produced with a higher conversion, a higher selectivity and a higher yield.

A support for Pd-based catalyst component may be selected from known supports without limitation. Examples of a support include silica (silicon dioxide), alumina, silica-alumina, carbon (activated charcoal); and oxides of zirconium, titanium, cerium, lanthanum, iron or the like, and composite oxides containing two or more types of these metals, and composite oxides containing one or more types of these metals and one or more types of other metals.

Among them, a preferable support for Pd-based catalyst component is silica. By using silica as a support, propane and/or butane can be produced with a higher selectivity and a higher yield without producing carbon dioxide as a by-product.

A silica support preferably has a specific surface area of 450 m²/g or more, more preferably 500 m²/g or more. By using a silica having a specific surface area within the above range, higher catalytic activity can be achieved and propane and/or butane can be produced with a higher conversion and a higher yield.

The upper limit of a specific surface area of a silica support is not particularly restricted, but is generally about 1000 m²/g.

A specific surface area of silica can be determined, for example, by a BET method using N₂ as an adsorption gas and a fully automatic measuring apparatus for specific surface area and pore distribution (e.g., ASAP2010, Shimadzu Corporation).

A Pd-based catalyst component may be a catalyst component in which other components, in addition to Pd, are supported on a support, as long as the desired effects of the catalyst would not be impaired.

A Pd-based catalyst component, in which Pd is supported on a support (e.g. silica), can be prepared by a known method such as an impregnation method and a precipitation method.

Some of Pd-based catalyst components must be activated by reduction treatment before use, including those containing Pd as an oxide, nitrate or chloride. In this invention, it is not necessarily required to activate a Pd-based catalyst component by reduction treatment in advance. The Pd-based catalyst component can be activated by reduction treatment of the catalyst for producing a liquefied petroleum gas of this invention, before the beginning of the reaction, after producing the catalyst by mixing a Pd-based catalyst component and a USY-type zeolite, and then molding the mixture. The conditions of the reduction treatment can be determined, depending on some factors such as the type of the Pd-based catalyst component, as appropriate.

A USY-type zeolite to be used may be selected from USY-type zeolites containing a metal such as alkali metals, alkaline earth metals and transition metals; USY-type zeolites ion-exchanged with these metals or the like; and USY-type zeolites on which these metals or the like are supported. But a preferable USY-type zeolite is a proton-type zeolite. By using a proton-type USY-type zeolite having a suitable acid strength and a suitable acidity (acid concentration), higher catalytic activity can be achieved, and propane and/or butane can be produced with a higher conversion and a higher selectivity. Alternatively, Pd and/or Pt supported USY-type zeolite can be preferably used.

A SiO₂/Al₂O₃ ratio of a USY-type zeolite is more preferably 5 or more, particularly preferably 15 or more. By using a USY-type zeolite with a SiO₂/Al₂O₃ ratio of 5 or more, particularly preferably 15 or more, the production amount of carbon monoxide and carbon dioxide as by-products can be more sufficiently reduced, and a higher selectivity of propane and butane can be achieved.

And, a SiO₂/Al₂O₃ ratio of a USY-type zeolite is preferably 70 or less, more preferably 50 or less, particularly preferably 40 or less, further preferably 25 or less. By using a USY-type zeolite with a SiO₂/Al₂O₃ ratio of 70 or less, further preferably 25 or less, a higher conversion of methanol and/or dimethyl ether can be achieved, and the production amount of methane as by-products can be more sufficiently reduced, and a higher selectivity of propane and butane can be achieved.

A mixed catalyst comprising a Pd-based catalyst component and a USY-type zeolite, that is a catalyst for producing a liquefied petroleum gas, is prepared by separately preparing a Pd-based catalyst component and a USY-type zeolite, and homogeneously mixing them, and then, if necessary, molding the mixture. A procedure of mixing and molding these catalyst components is not particularly limited, but is preferably a dry method. When mixing and molding these catalyst components by a wet method, there may occur a compound transfer between these catalyst components, for example, neutralization due to transfer of a basic component in a Pd-based catalyst component to an acidic site in a USY-type zeolite, leading to the change of a property optimized for each function of these catalyst components, and the like. Examples of a molding method of a catalyst include an extrusion and a tablet-compression.

A catalyst for producing a liquefied petroleum gas may comprise other additive components as long as its intended effect would not be impaired. For example, any of the above catalysts may be diluted with quartz sand and then used.

When the reaction is conducted with a fixed bed, in the catalyst layer comprising a catalyst for producing a liquefied petroleum gas, the composition may change in regard to the direction of flowing of the starting gas. The catalyst layer may consist of, for example, a former catalyst layer comprising a zeolite largely, and a latter catalyst layer comprising a catalyst component in which an olefin-hydrogenation catalyst component is supported on a support such as silica, or a methanol synthesis catalyst component largely, in the direction of flowing of the starting gas.

In the liquefied petroleum gas production process, a paraffin comprising propane or butane, preferably propane, as a main component is produced by reacting at least one selected from the group consisting of methanol and dimethyl ether with hydrogen using at least one of the catalysts for producing a liquefied petroleum gas as described above.

The reaction can be conducted in a fixed bed, a fluidized bed or a moving bed. The reaction conditions such as a composition of a starting gas, a reaction temperature, a reaction pressure and a contact time with a catalyst can be appropriately determined, depending on a kind of a catalyst to be used, and the like. For example, the LPG production reaction may be carried out under the following conditions.

As described above, a gas fed into a reactor is the crude methanol obtained in the above methanol production process, or the crude dimethyl ether obtained in the above dimethyl ether production process. A gas fed into a reactor may contain both methanol and dimethyl ether. In this case, a ratio of methanol to dimethyl ether is not particularly limited, and a gas having any of ratios of methanol to dimethyl ether can be used. Hydrogen may be added to the crude methanol or the crude dimethyl ether, as necessary.

In the light of achieving a higher catalytic activity, a reaction temperature is preferably 300° C. or higher, more preferably 320° C. or higher. In the light of achieving a higher selectivity for a hydrocarbon and a higher selectivity for propane and butane, as well as a long catalyst life, a reaction temperature is preferably 470° C. or lower, more preferably 450° C. or lower, particularly preferably 400° C. or lower.

In the light of achieving a higher activity and good operability of an apparatus, a reaction pressure is preferably 0.1 MPa or higher, more preferably 0.15 MPa or higher. In the light of economical efficiency and safety, a reaction pressure is preferably 3 MPa or lower, more preferably 2.5 MPa or lower.

Furthermore, according to this invention, LPG can be produced under a further lower pressure. Specifically, LPG can be produced from at least one selected from the group consisting of methanol and dimethyl ether, and hydrogen under a pressure of lower than 1 MPa, particularly 0.6 MPa or lower.

A gas space velocity of methanol and/or dimethyl ether is preferably 1500 hr⁻¹ or more, more preferably 1800 hr⁻¹ or more, in the light of economical efficiency. In addition, a gas space velocity of methanol and/or dimethyl ether is preferably 60000 hr⁻¹ or less, more preferably 30000 hr⁻¹ or less, in the light of achieving a higher activity and a higher selectivity for propane and butane.

A gas fed into a reactor can be dividedly fed to the reactor so as to control a reaction temperature.

The reaction can be conducted in a fixed bed, a fluidized bed, a moving bed or the like, and can be preferably selected, taking both of control of a reaction temperature and a regeneration method of the catalyst into account. For example, a fixed bed may include a quench type reactor such as an internal multistage quench type, a multitubular type reactor, a multistage type reactor having a plurality of internal heat exchangers or the like, a multistage cooling radial flow type, a double pipe heat exchange type, an internal cooling coil type, a mixed flow type, and other types of reactors.

When used, a catalyst for producing a liquefied petroleum gas can be diluted with silica, alumina or an inert and stable heat conductor for controlling a temperature. In addition, when used, a catalyst for producing a liquefied petroleum gas can be applied to the surface of a heat exchanger for controlling a temperature.

A reaction product gas thus produced (a lower-paraffin-containing gas) comprises a hydrocarbon containing propane or butane as a main component. In the light of liquefaction properties, it is preferable that the total content of propane and butane is higher in a lower-paraffin-containing gas. Furthermore, a lower-paraffin-containing gas produced preferably contains more propane in comparison with butane, in the light of inflammability and vapor pressure properties.

A lower-paraffin-containing gas produced generally comprises water; a low-boiling component having a lower boiling point or a lower sublimation point than the boiling point of propane; and a high-boiling component having a higher boiling point than the boiling point of butane. Examples of a low-boiling component include carbon monoxide and carbon dioxide; hydrogen, which is an unreacted starting material; and ethane and methane, which are by-products. Examples of a high-boiling component include high-boiling paraffins (e.g., pentane, hexane and so on), which are by-products.

Thus, water, a low-boiling component and a high-boiling component are, as necessary, separated from a lower-paraffin-containing gas produced, so as to obtain a liquefied petroleum gas (LPG) comprising propane or butane as a main component. If necessary, methanol and/or dimethyl ether, which are unreacted starting materials, are also separated from a lower-paraffin-containing gas by a known method.

Separation of water, a low-boiling component or a high-boiling component can be conducted in accordance with a known method.

Water can be separated by, for example, liquid-liquid separation.

A low-boiling component can be separated by, for example, gas-liquid separation, absorption separation or distillation; more specifically, gas-liquid separation at an ambient temperature under increased pressure, absorption separation at an ambient temperature under increased pressure, gas-liquid separation with cooling, absorption separation with cooling, or combination thereof. Alternatively, for this purpose, membrane separation or adsorption separation can be conducted, or these in combination with gas-liquid separation, absorption separation or distillation can be conducted. A gas recovery process commonly employed in an oil factory (described in “Oil Refining Processes”, ed. The Japan Petroleum Institute, Kodansha Scientific, 1998, pp. 28-32) can be applied to separation of a low-boiling component.

A preferable method of separation of a low-boiling component is an absorption process where a liquefied petroleum gas comprising propane or butane as a main component is absorbed into an absorbent liquid such as a high-boiling paraffin gas having a higher boiling point than butane, and a gasoline.

A high-boiling component can be separated by, for example, gas-liquid separation, absorption separation or distillation.

The separation conditions may be determined as appropriate in accordance with a known method.

For consumer use, it is preferable that a content of a low-boiling component in the LPG is reduced to 5 mol % or less (including 0 mol %) by separation, for example, in the light of safety in use.

If necessary, the gas may be pressurized and/or cooled so as to obtain a liquefied petroleum gas.

The component(s) separated from the lower-paraffin-containing gas can be removed outside the system, and can be recycled to any process as described above. For example, carbon monoxide and hydrogen separated in this step can be recycled as a starting material for the methanol production process or the dimethyl ether production process. Methanol, dimethyl ether and hydrogen separated in this step can be recycled as a starting material for the lower-paraffin production process.

For the purpose of recycling a separated component, a known technique, e.g., appropriately providing a recycle line with a pressurization means can be employed.

The total content of propane and butane in the LPG thus produced may be 90% or more, more preferably 95% or more (including 100%) on the basis of carbon. And a content of propane in the LPG produced may be 50% or more, more preferably 60% or more, particularly preferably 65% or more (including 100%) on the basis of carbon. Thus, according to this invention, LPG having a composition suitable for a propane gas, which is widely used as a fuel for household and business use, can be produced.

Thus, according to this invention, LPG containing propane or butane as a main component is produced from a carbon-containing starting material such as a natural gas, or from a synthesis gas, via methanol and/or dimethyl ether.

### EXAMPLES

The following will describe the present invention in more detail with reference to Examples. However, the present invention is not limited to these Examples.

### Example 1

### Preparation of the Catalyst for Producing a Liquefied Petroleum Gas

A mechanically pulverized proton-type ZSM-5 with a Si/Al ratio (atomic ratio) of 20, produced by Tosoh Corporation, was used as a zeolite for a support of an olefin-hydrogenation catalyst component. And, 0.5 wt % of Pd was supported on the ZSM-5 by an ion exchange method as follows.

First, 0.0825 g of palladium chloride (purity:>99 wt %) was dissolved in 10 mL of a 12.5 wt % aqueous ammonia solution at 40 to 50° C. And then, 150 mL of ion-exchanged water was added to the resulting solution to obtain a Pd-containing solution. 10 g of ZSM-5 zeolite was added to the obtained Pd-containing solution, and the mixture was heated and stirred at 60 to 70° C. for 6 hours. After the ion-exchange process, the resulting material was repeatedly filtrated and washed with ion-exchanged water until no chloride ions were observed in a filtrate.

Then, the Pd ion-exchanged ZSM-5 was dried at 120° C. for 12 hours, and calcined at 500° C. in an air for 2 hours. Subsequently, it was mechanically pulverized, and then molded by a tablet-compression and sized to give a granular catalyst for producing a liquefied petroleum gas (Pd-ZSM-5) having an average particle size of 1 mm.

In a tubular reactor was placed the prepared catalyst for producing a liquefied petroleum gas, and the catalyst was reduced under a hydrogen stream at 400° C. for 3 hours before the beginning of the reaction.

(Production of LPG)

Methanol synthesis reaction was carried out using the synthesis gas having the composition of H₂:CO:CO₂=66.6:31.7:1.7 (molar ratio), to give a methanol-containing gas (crude methanol) having the following composition:

methanol:5.49 mol %; dimethyl ether:0.04 mol %; methyl formate:0.04 mol %; ethanol:0.02 mol %; methane:0.04 mol %; CO:29.27 mol %; CO₂:1.83 mol %; H₂:62.19 mol %; H₂O:1.10 mol %.

The crude methanol thus obtained was passed through the catalyst layer consisting of the above catalyst for producing a liquefied petroleum gas, at a reaction temperature of 350° C., a reaction pressure of 2.1 MPa and a gas space velocity of methanol of 2000 hr⁻¹ (W/F=9.0 g·h/mol) to carry out the LPG production reaction. Gas chromatographic analysis of the product indicated that all carbon-containing compounds except methane, CO and CO₂ was converted into hydrocarbons, 1.2% of CO was converted into hydrocarbons. The carbon number distribution of hydrocarbons thus obtained (including methane in the crude methanol) was as follows;

| | |
|---|---|
| C1 (methane): | 3.6 [C-mol %], |
| C2 (ethane): | 25.6 [C-mol %], |
| C3 (propane): | 35.3 [C-mol %], |
| C4 (butane): | 16.2 [C-mol %], |
| C5 (pentane): | 9.8 [C-mol %], |
| C6 (hexane): | 7.6 [C-mol %], |
| C7 (heptane): | 1.9 [C-mol %]. |

The produced hydrocarbon gas contained propane and butane in 51.5% on the basis of carbon. The result shows that the hydrocarbon gas containing propane or butane as a main component can be produced from the crude methanol without purification.

### Example 2

The catalyst prepared in the same way as Example 1 (Pd-ZSM-5) was used as a catalyst for producing a liquefied petroleum gas.

(Production of LPG)

Dimethyl ether synthesis reaction was carried out using the synthesis gas having the composition of H₂:CO=50:50 (molar ratio), to give a dimethyl ether-containing gas (crude dimethyl ether) having the following composition:

dimethyl ether:16.41 mol %; methanol:2.53 mol %; methyl formate:0.08 mol %; ethanol:0.01 mol %; methane:0.08 mol %; CO: 33.67 mol %; CO₂: 15.99 mol %; H₂:29.46 mol %; H₂O:1.77 mol %.

The crude dimethyl ether thus obtained was passed through the catalyst layer consisting of the above catalyst for producing a liquefied petroleum gas, at a reaction temperature of 350° C., a reaction pressure of 2.1 MPa and a gas space velocity of dimethyl ether of 2000 hr⁻¹ (W/F=9.0 g·h/mol) to carry out the LPG production reaction. Gas chromatographic analysis of the product indicated that all carbon-containing compounds except methane, CO and CO₂ was converted into hydrocarbons, 6.1% of CO was converted into hydrocarbons. The carbon number distribution of hydrocarbons thus obtained (including methane in the crude dimethyl ether) was as follows;

| | |
|---|---|
| C1 (methane): | 3.5 [C-mol %], |
| C2 (ethane): | 25.8 [C-mol %], |
| C3 (propane): | 35.7 [C-mol %], |
| C4 (butane): | 17.1 [C-mol %], |
| C5 (pentane): | 8.9 [C-mol %], |
| C6 (hexane): | 6.8 [C-mol %], |
| C7 (heptane): | 2.2 [C-mol %]. |

The produced hydrocarbon gas contained propane and butane in 52.8% on the basis of carbon. The result shows that the hydrocarbon gas containing propane or butane as a main component can be produced from the crude dimethyl ether without purification.

### INDUSTRIAL APPLICABILITY

As described above, according to this invention, a hydrocarbon containing propane or butane as a main component, i.e., a liquefied petroleum gas (LPG) can be economically produced from a carbon-containing starting material such as a natural gas, or from a synthesis gas, via methanol and/or dimethyl ether.

1: A process for producing a liquefied petroleum gas, comprising:
(i) a step of producing methanol wherein crude methanol containing methanol, hydrogen and at least one selected from the group consisting of carbon monoxide and carbon dioxide is produced from a synthesis gas using a methanol synthesis catalyst; and
(ii) a step of producing a liquefied petroleum gas wherein a liquefied petroleum gas containing propane or butane as a main component is produced from the crude methanol, which is fed from the step of producing methanol without purification, using a catalyst for producing a liquefied petroleum gas.

2: A process for producing a liquefied petroleum gas, comprising:
(I) a step of producing a synthesis gas from a carbon-containing starting material and at least one selected from the group consisting of H₂O, O₂ and CO₂;
(II) a step of producing methanol wherein crude methanol containing methanol, hydrogen and at least one selected from the group consisting of carbon monoxide and carbon dioxide is produced from the synthesis gas using a methanol synthesis catalyst; and
(III) a step of producing a liquefied petroleum gas wherein a liquefied petroleum gas containing propane or butane as a main component is produced from the crude methanol, which is fed from the step of producing methanol without purification, using a catalyst for producing a liquefied petroleum gas.

3: A process for producing a liquefied petroleum gas, comprising:
(i) a step of producing dimethyl ether wherein crude dimethyl ether containing dimethyl ether, hydrogen and at least one selected from the group consisting of carbon monoxide and carbon dioxide is produced from a synthesis gas using a dimethyl ether synthesis catalyst; and
(ii) a step of producing a liquefied petroleum gas wherein a liquefied petroleum gas containing propane or butane as a main component is produced from the crude dimethyl ether, which is fed from the step of producing dimethyl ether without purification, using a catalyst for producing a liquefied petroleum gas.

4: A process for producing a liquefied petroleum gas, comprising:
(I) a step of producing a synthesis gas from a carbon-containing starting material and at least one selected from the group consisting of H₂O, O₂ and CO₂;
(II) a step of producing dimethyl ether wherein crude dimethyl ether containing dimethyl ether, hydrogen and at least one selected from the group consisting of carbon monoxide and carbon dioxide is produced from the synthesis gas using a dimethyl ether synthesis catalyst; and
(III) a step of producing a liquefied petroleum gas wherein a liquefied petroleum gas containing propane or butane as a main component is produced from the crude dimethyl ether, which is fed from the step of producing dimethyl ether without purification, using a catalyst for producing a liquefied petroleum gas.

5: A process for producing a liquefied petroleum gas according to claim 3, wherein the dimethyl ether synthesis catalyst comprises a methanol synthesis catalyst component and a methanol dehydration catalyst component.

6: A process for producing a liquefied petroleum gas according to claim 1, wherein the catalyst for producing a liquefied petroleum gas comprises an olefin-hydrogenation catalyst component and a zeolite.

7: A process for producing a liquefied petroleum gas according to claim 6, wherein the olefin-hydrogenation catalyst component is at least one selected from the group consisting of Fe, Co, Ni, Cu, Zn, Ru, Rh, Pd, Ir and Pt.

8: A process for producing a liquefied petroleum gas according to claim 7, wherein the catalyst for producing a liquefied petroleum gas is a catalyst in which Pd and/or Pt is supported on ZSM-5 or USY-type zeolite.

9: A process for producing a liquefied petroleum gas according to claim 8, wherein a Si/Al ratio (atomic ratio) of the ZSM-5 is 20 to 100, or a SiO₂/Al₂O₃ ratio of the USY-type zeolite is 5 to 70.

10: A process for producing a liquefied petroleum gas according to claim 8, wherein the total amount of supported Pd and/or Pt in the catalyst for producing a liquefied petroleum gas is 0.005 to 5% by weight.

11: A process for producing a liquefied petroleum gas according to claim 7, wherein the catalyst for producing a liquefied petroleum gas comprises a Pd-based catalyst component in which Pd is supported on a support; and a USY-type zeolite.

12: A process for producing a liquefied petroleum gas according to claim 1, wherein a ratio (by weight) of the Pd-based catalyst component to the USY-type zeolite is 0.1 to 1.5 (Pd-based catalyst component/USY-type zeolite).

13: A process for producing a liquefied petroleum gas according to claim 1, wherein the amount of supported Pd in the Pd-based catalyst component is 0.1 to 5% by weight.

14: A process for producing a liquefied petroleum gas according to claim 1, wherein the support for the Pd-based catalyst component is silica.

15: A process for producing a liquefied petroleum gas according to claim 1, wherein the USY-type zeolite has a SiO₂/Al₂O₃ ratio of 5 to 70.
